(19) United States
(12) Patent Application Publication
(10) Pub. No.: US 2009/0203548 A1
(43) Pub. Date: Aug. 13, 2009

(54) Complex able to detect an analyte, method for its preparation and uses thereof

(76) Inventor: Roberto Burioni, Roma (IT)

Correspondence Address: JUNEAU PARTNERS, P.O. BOX 2516, ALEXANDRIA, VA, 22301, US

(21) Appl. No.: 11/659,460

(22) PCT Filed: Aug. 9, 2005
(86) PCT No.: PCT/IT05/00488

§ 371 Date: Apr. 22, 2009

(30) Foreign Application Priority Data
Aug. 10, 2004 (IT) RM2004A000406

(51) Int. Cl. C40B 40/08; C12Q 1/68; C12Q 1/70

(52) U.S. Cl. 506/17; 435/6; 435/5

(57) ABSTRACT

A complex able to detect an analyte (CRA) comprising a particle expressing on its outer surface a compound having specific binding capability (CDCLS) for the analyte and stably including at least one nucleic acid reporter sequence being univocally associated to the CDCLS; process for its construction and uses thereof.

### TECHNICAL FIELD

The present invention relates to a method to detect an analyte by means of affinity and subsequent amplification of nucleic acids associated to a compound having specific binding capability (CDCLS) with respect to the analyte. The compound having specific binding capability can be a specific antibody (being either a whole monoclonal or purified antibody, a Fab fragment, an antibody in single chain form, or a synthetic derivative), or a non antibody peptide, or any other specific reagent. All these compounds shall hereinafter be called compounds having specific binding capability, CDCLS.

In greater detail, the invention consists of a complex able to detect an analyte (CRA) comprising the CDCLS and a nucleic acid of defined sequence incorporated inside a particle, i.e. a recombinant virus particle, which expresses the CDCLS on its outer surface. The binding of the CDCLS to the analyte is detected, with considerable simplicity, sensitivity and specificity, by amplification and/or detection of the nucleic acid.

The invention also consists of a method for the construction of collection of complexes able to detect an analyte, by recombinant procedures to get particles, i.e. a recombinant virus particle expressing on the surface the CDCLS and containing a nucleic acid reporter sequence.

The invention enables to generate CRA in an economical, fast, reliable and safe fashion with respect to existing technologies and it will allow the execution of single or multiple dosages of analytes in a simple fashion and with a very considerable reduction in the costs.

### BACKGROUND OF THE INVENTION

The introduction of quantitative immunological assays has allowed the precise quantification of a very high number of analytes, by the direct or indirect measure of marked antibodies bound to the analytes, or by evaluating the analytes' ability to inhibit the formation of a marked antibody-analyte complex. The marking of the antibody or of the analyte can be obtained using radioactive isotopes (as in radio-immunological and radio-immunometric dosages), using enzymes able to be revealed by colorimetry, or using secondary antibodies marked with the above methods.

The sensitivity of a system of this kind is given by first, the affinity of the binding of the antibody or of another compound with the analyte. Secondly, a limiting element of primary importance is the ability of the detection system to reveal reduced quantity of antibodies (or other compound) bound to the analyte, when the analyte is present in extremely low quantities. The systems that use enzymatic and fluorescent markings solve numerous drawbacks of radioisotopic labelling, but at the price of a diminished sensitivity of the system.

Numerous strategies have been devised (Baldo, Tovey et al. 1986; Hauri and Bucher 1986; Ruan, Hashida et al. 1986; Wedege and Svenneby 1986; Vogt, Phillips et al. 1987; Graves 1988; Tovey, Ford et al. 1989; Bodmer and Tiefenauer 1990; Pruslin, To et al. 1991; Rodda and Yamazaki 1994) and very encouraging results have been obtained when the detection of the binding of the antibody to the analyte (and hence the indirect determination of the presence of the analyte) was performed by the detection of a nucleic acid bound to the antibody.

There are numerous methods that enable to reveal the presence of a particular nucleic acid which, once bound to an antibody, can be used to detect the presence of the antibody itself and hence to the analyte in question. Among the methods able to reveal the presence of a nucleic acid, worthy of mention is molecular hybridisation, either simple or using polymeric probes (U.S. Pat. No. 4,888,269, WO89/03891). A signal is obtained by molecular hybridisation of a nucleic acid, modified as needed, with a second complementary nucleic acid able to bind specifically to the sought nucleic acid and able to emit a signal. The method for amplifying nucleic acids by polymerase chain reaction (PCR) has allowed to develop simple and sensitive assays able to recognise, for the most disparate uses, the presence in a sample of a nucleic acid with defined sequence (Sanger and Coulson 1975; Maxam and Gilbert 1977; Li, Cui et al. 1990). This capability has also been utilized in the field of determining the presence of analytes using antibody molecules in the so-called immuno-PCR (U.S. Pat. No. 5,665,539). In this technology, a biotinylated DNA is linked by a streptavidin bridge to a biotinylated antibody and a segment of the DNA is amplified by PCR. Therefore, the detection of an analyte is obtained by revealing the amplification of the DNA on agarose gel (Sano, Smith et al. 1992; Zhou, Fisher et al. 1993). Other researchers have developed a chimeric molecule composed by a fusion between protein A (able to bind the antibodies) and streptavidin (able to bind the biotin and hence the biotinylated DNA) (Sano and Cantor 1991; Sano, Smith et al. 1992; Zhou, Fisher et al. 1993). A similar method is also described in WO 9315229. The use of a very sensitive detection system that facilitates quantification by rugged, proven methods could solve the problem of aspecific binding of the antibodies as well as the aspecific activation of the detection system. These drawbacks worsen the signal-to-noise ration of the analyte dosage system, preventing the use of antibodies having very high affinity. These antibodies are obtainable nowadays with sophisticated molecular maturation methods and would allow the detecting of a very small number of molecules. However due to the inadequate signal-to-noise ratio not of the primary binding system (antibodies) but because of the inadequacy of the binding detection system, their use is not possible.

The possibility of dosing an analyte by an antibody (or CDCLS) bound to a nucleic acid and the subsequent demonstration of the binding by amplification and detection (also quantitative) of the nucleic acid bound to the antibody (or CDCLS) appears extremely promising. Indeed, this methodology combines the molecular recognition capability of the antibodies with the reliability, flexibility, sensitivity and rapidity of amplification by PCR method which has allowed, so far, to detect 600 molecules of antigen immobilised with antibodies of conventional affinity (Sano and Cantor 1991; Sano, Smith et al. 1992; Zhou, Fisher et al. 1993). This new technology, however, is not free from problems. First of all, the use of a streptavidin-biotin (or streptavidin-protein A bridge) to bind the reporter nucleic acid to the antibody does not allow the use of different antibodies for the simultaneous dosage of multiple analytes. The non covalent nature of the bond between biotin and streptavidin is such that the nucleic acid marking the antibodies can be switched, thereby making the assay totally aspecific. Moreover, in all these approaches the antibody-DNA complex is formed in situ while the analysis is carried out. This can generate an additional variability in the reaction, as well as a complication of the method. It thus becomes necessary to provide a system that binds in a stable and irreversible fashion the antibody (or CDCLS) to the nucleic acid that marks it (detectable in specific fashion). Such system could not only render simpler, more rapid and more economical the determination of a single analyte, but could also achieve the fundamental goal of dosing numerous analytes in a single assay. Indeed such system would benefit from existing possibility of devising multiplex amplification systems that allow amplification, with common primers, of different fragments of DNA which can then be differentiated thanks to their sequence or their molecular weight.

An attempt to solve this problem was made by Hendrickson et al (Hendrickson, Truby et al. 1995, U.S. Pat. No. 6,511,809) who marked antibodies with a DNA fragment bound in a covalent, and hence extremely stable, fashion. In this approach the analyte-specific antibody (or CDCLS) and the DNA modified at the 5′ end are activated independently by heterobifunctional cross-linking agents. Subsequently, the activated antibodies and DNA are bound in a single spontaneous reaction. The binding of the antibodies to the analyte-specific DNA complex is lastly demonstrated by a PCR able to amplify the DNA (or the different DNAs bound to the antibodies that are used simultaneously to dose the different analytes) which then, in this specific case, is demonstrated by means of agarose gel. For the detection of multiple analytes, different DNAs amplified by the same pair of primers thanks to appropriate sequences inserted in strategic position, are then differentiated by agarose gel due to the different size of the fragment inserted between the sequences recognised by the pair of primers. The method has been found to be extremely effective both in terms of sensitivity in the dosage of single analytes, and in terms of ability to dose multiple analytes. However, the described method appears quite far from what would ideally be desired for a practical use. The methods used for the production of activated antibodies (and of activated DNA) are long and very expensive both in terms of reagents, and labour. Moreover, the reagents used are often hazardous, easily perishable, and strongly pollutant. Indeed, the DNA has to be synthesised every time in large quantities, then it has to be activated with N-succinimidyl S-acetilthioacetate, immediately applied to a column for Sephadex® chromatography, eluted with spectrophotometer monitoring, concentrated twice and lastly preserved with particular cautions. The antibodies must be activated with other reagents and they also require numerous complicated contrivances for their preparation. The reaction is so delicate and unstable that the authors themselves (Hendrickson, Truby et al. 1995) indicate that it is in fact necessary to synchronise the delicate preparation of the two reagents (activated DNA and activated antibody) with imaginable practical difficulties, since the active groups can be deactivated in aqueous solution. Moreover, the conjugation between antibodies and DNA requires a complicated procedure and the use of complex and expensive machinery. Lastly, the antibody-DNA complex must be purified again by HPLC and other complicated procedures to separate non conjugated substances. Given the complexity of the reaction, it is not surprising that the DNA/antibodies ratio measured by the authors themselves is extremely variable depending on the different preparations (Hendrickson, Truby et al. 1995). This variability imposes to perform standard curves for each individual batch or reagent, with obvious practical limitations.

In conclusion, prior art provides reagents not as specific as desired and obtainable by extremely complex procedures, to be repeated for each individual CDCLS (or antibody) preparation. In other words, the set up of a system for detecting a big amount of analytes (thousands), theoretically possible, requires the repetition of the complicated procedure as many times as there are analytes to be detected. Moreover, the produced calibrated reagent has non reproducible features, resulting to be not applicable to all of antibody batches. Lastly, in the methods of prior art, given the different length of the DNA, a difference in amplification efficiency is likely, altering the efficiency and accuracy of the analyte dosage.

### DESCRIPTION OF THE INVENTION

It is an object of the invention a complex able to detect an analyte (CRA) comprising a particle expressing on its outer surface a compound having specific binding capability (CDCLS) for the analyte and stably including at least one nucleic acid reporter sequence being univocally associated to the CDCLS. Preferably the particle is a recombinant particle, more preferably a recombinant virus particle, most preferably a recombinant bacterial phage particle.

In a preferred embodiment the nucleic acid reporter sequence encodes for a detectable marker, preferably a phosphatase or a beta-galactosidase.

In alternative embodiment the nucleic acid reporter sequence is flanked at its 5′ end by a first primer sequence, and at its 3′ end, by a second primer sequence.

In a preferred embodiment the CDCLS is an antibody, or a functional fragment thereof obtained by synthetic or recombinant procedures, or a bispecific antibody. Alternatively the CDCLS is a non antibody protein, a peptide, even in multimeric form and/or made by modified or non natural amino acids.

It is a further object of the invention a recombinant or combinatorial library comprising a collection of the complexes of the invention wherein each CDCLS is associated to a different nucleic acid reporter sequence. Preferably the first primer sequence and the second primer sequence are each hybridisable to a first primer and to a second primer under high stringency conditions, respectively.

It is a further object of the invention a process for constructing the complex of the invention comprising the steps of:

a) inserting into an host cell an appropriate recombinant vector comprising coding sequences for the CDCLS linked to appropriate sequences to direct its expression on the outer surface of a recombinant virus particle;
b) transforming cells as obtained in a) with a packageable genome containing the nucleic acid reporter sequence, and
c) infecting said transformed cells with a helper virus able to rescue a recombinant virus particle expressing on its outer surface the CDCLS and stably including at least one nucleic acid reporter sequence.

It is a further object of the invention a process for constructing the complex of the invention comprising the steps of:

a) transforming an host cell an appropriate recombinant viral vector comprising. i) coding sequences for the CDCLS linked to appropriate sequences to direct its expression on the outer surface of a recombinant virus, ii) nucleic acid sequences allowing the encapsulation of the vector inside the recombinant virus particle and iii) the nucleic acid reporter sequence;
b) infecting said transformed cells with a helper virus able to rescue a recombinant virus particle expressing on its outer surface the CDCLS and stably including at least one nucleic acid reporter sequence.

In a preferred embodiment the appropriate recombinant viral vector consists in a collection of different vectors, each one comprising a given CDCLS coding sequence univocally associated to a given nucleic acid reporter sequence.

It is a further object of the invention a method for detecting an analyte in a sample comprising the steps of:

a) incubating the sample with a solid phase specific for the analyte in such conditions that, if present, the analyte binds to the solid phase;
b) incubating the solid phase whereto is bound the analyte, if present, with the CRA of the invention in conditions that, if present, the analyte binds to the CDCLS of the CRA;
c) separating the solid phase-analyte-CRA complexes from non bound CRAs;
d) detecting the reporter sequences present in the solid phase-analyte-CRA complex.

Preferably the detection of the reporter sequences is made by an amplification thereof.

It is a further object of the invention a kit for detecting an analyte in a sample comprising the complex of the invention.

The invention relates to the set up of a complex able to detect an analyte (CRA) constituted by: a virus expressing on its outer surface a compound having specific binding capability (CDCLS) for the analyte and stably including in its interior a nucleic acid of defined sequence. The binding of the CDCLS to the analyte is detected, with considerable simplicity, sensitivity and specificity, by the detection of the nucleic acid contained in the phage. The latter is detected by amplification and/or by any method for detecting nucleic acid known to those skilled in the art.

In one embodiment the virus is a bacterial virus, preferably it is a filamentous phage, more preferably the M13 phage.

The invention enables to generate CRA in an economical, fast, reliable and safe fashion with respect to existing technologies and the execution of single or multiple dosages of analytes in a simple fashion and with a very considerable reduction in the costs for the production of the CRA.

As a non limiting embodiment, the author has set up an M13 filamentous phage that exposes on its surface, bound to the cp3 phage protein, but other phage proteins are equally usable.

The engineered M13 filamentous phage is produced by infecting with a phage helper, a bacterial cell already modified by inserting the necessary genes on the chromosome, or a bacterial cell transformed with an appropriately modified vector in order to allow the bacterium to produce, constitutively or in an inducible fashion, a recombinant chimeric protein constituted by a fragment of the heavy chain of the antibody (CDCLS), fused to a region of a phage protein. The fusion protein is engineered in such a way as not to compromise the ability of the protein to be incorporated in the structure of the phage, since thanks to the infection of a phage helper a productive infection occurs in the cell, leading to the production of phages that contain the antibody (CDCLS) on its surface. The bacterial cell also contains a phage that, thanks to the presence of an whole phage replication origin, will constitute the genome of the phage produced by this cell. Since the phage is a stable structure, linked in equally stable fashion to an antibody (or CDCLS), and since the genome of the phage is contained in stable fashion inside the phage itself, by this method a stable binding will be achieved between the antibody (or CDCLS), exposed on the surface of the phage, and the DNA that will be used to detect the bond, contained inside the phage. The DNA of the phagemid (which will become the genome of the phage) was modified in such a way as not to compromise either phage production or the ability of the phage-antibody (CDCLS) complex to bind the antigen with specificity.

The sequence inserted in the genome of the phage is advantageously constituted by two conserved terminal primers (primer A and primer B) and by a central reporter sequence being different for each CDCLS, according to the following organisation: primer A-reporter sequence [label]-primer B.

The coding genes for the antibody (or CDCLS) fused to the phage protein can be contained in the same phagemid that contains the reporter sequence. However, it is possible to construct a bacterial cell that contains the coding genes for the CDCLS in a different genic structure in order to use the phagemid exclusively for the reporter sequence. The use of a single reporter sequence per phagemid is described here, but it is also possible to use multiple reporter sequences in the same phagemid (whether or not it contains the CDCLS genes), in order to detect the binding of each CDCLS. The detection is performed through multiple hybridisation or amplification reactions with quantitative PCR, to improve the sensitivity and the specificity of the analyte detection system.

In one embodiment, the possibility of using as phage helper viruses lacking the protein that is used to generate the fusion protein with the CDCLS further allows to produce “superphages” that lack the protein used for the protein-CDCLS fusion in the wild form. These “superphages” are not able to infect but, since they contain exclusively the protein in the protein-antibody form (or CDCLS) on the surface, they can be used to improve the efficiency of the technology. Examples of such superphages are described in Dubel S. Nature Biotechnology.

Whereas with methods that currently represent the “state of the art”, the production of the stable conjugate DNA-CDCLS is extremely difficult, once a recombinant host cell (e.g. E. Coli) is produced as described in the present invention, it contains the coding sequence for the CDCLS fused to that of the phage protein under the control of appropriate promoter sequences. Therefore it is sufficient to infect the bacterium with a phage helper, and to let it grow according to ordinary classic virology procedures. It is then possible to separate the bacteria from the supernatant (which contains the CDCLS-phage-DNA (CAFD) complex) by means of known low-speed centrifuging techniques. The phages are then precipitated by means of sodium chloride and polyethylene glycol. The production of the CDCLS-phage-DNA (CAFD) complex can be repeated without any difficulty, and up scaling is very simple using current fermentation techniques without any environmental, chemical or infective risk. Obtaining the CDCLS-phage-DNA (CAFD) complex does not require either costly equipment, or specialised labour, or many hours of work.

The method can also use vectors mutated in the region of insertion of extraneous sequences for the construction of libraries of CDCLS by phage exposure, in order simultaneously to obtain both the CDCLS (which can be used in current methods for its evaluation) and the CDCLS-phage-DNA (CAFD) complex ready for use in this new format. In other words, vectors that have the label sequences (Primer A-different label for every Ab-Primer B) with mutations already present can be used. In this case, the antibody would be cloned in a vector that already contains a label region—between two primers—that contains at the origin a sequence where mutations were introduced and hence every different antibody of a repertory is already with its label sequence, which need only be determined.

The availability of a CDCLS stably fused to a pre-defined DNA sequence allows to design systems for the quantitative dosage of an unlimited number of analytes in the same assay. The reporter sequence can be designed in such a way as to use the same pair of primers for the amplification of all reporter DNAs present in the different CDCLS-phage-DNA (CAFD) complexes used for the detection of different analytes. The different reporter DNAs, together with a quantitative standard, are then distinguished and quantified using the sequence of the DNA included between the pair of primers, which is different for each CDCLS. The presence of multiple reporter sequences considerably increases the signal/noise ratio, greatly improving the performance of the analyte detection system.

The DNA that is incorporated in the CDCLS-phage-DNA (CAFD) complex is not modified and hence can be amplified using primers conjugated to fluorochromes or to biotin, rendering the detection and the quantification of the amplified sequences extremely simple.

In addition to the use of real time PCR in multiplex (taking advantage of the identical primers for all CDCLS-phage-DNA (CAFD) complexes and the variable internal sequences) this system can be used together with chips whereon are fixed the DNA sequences complementary to the inner variable region that is amplified with a marked primer, and thus easily detectable.

The invention will now be illustrated in its explicatory but non limiting examples with reference to the following figures:

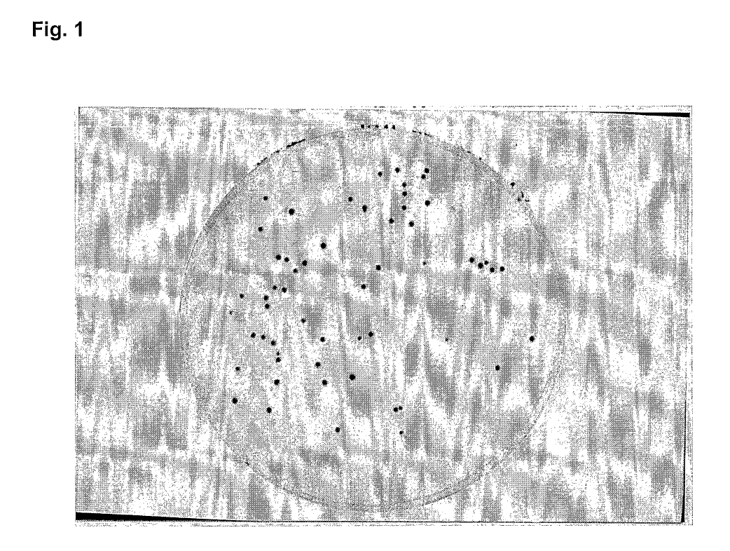

FIG. 1: Bacterial cells containing pComb3/white (white colonies) and pComb3/green (green colonies) plated on semi-solid medium TPA/MG and observed after 18 hours at 37° C.

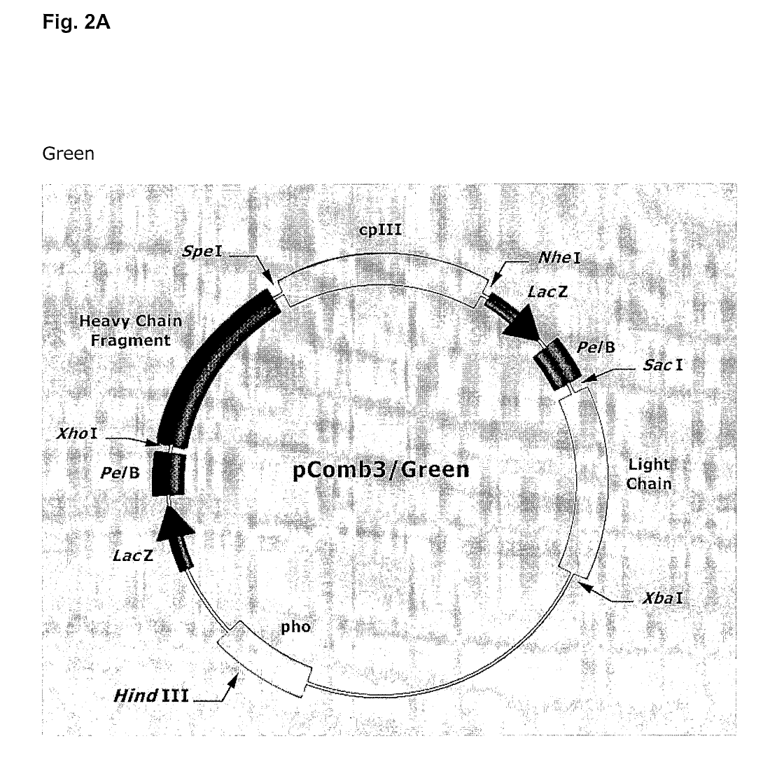

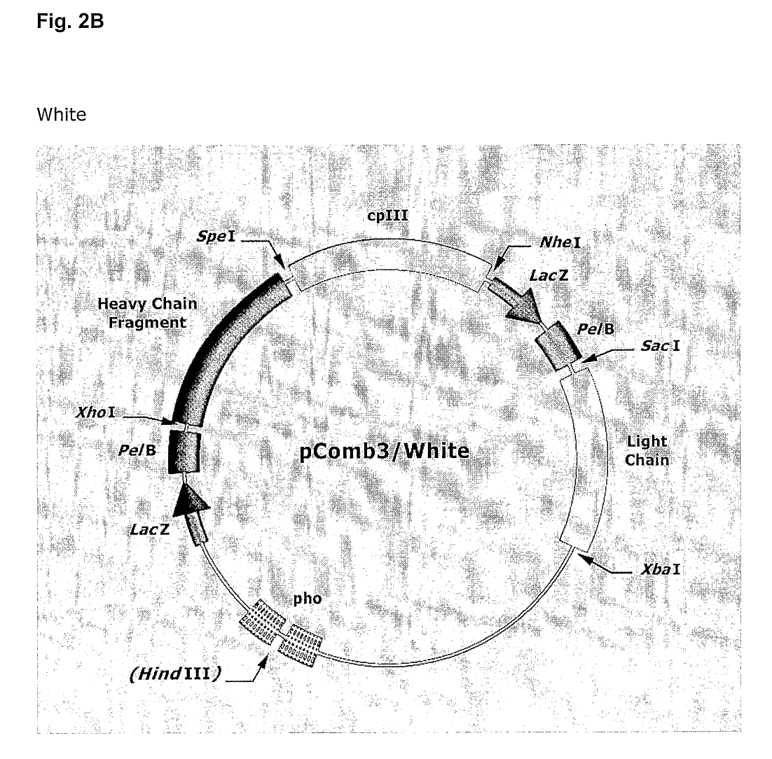

FIG. 2: Schematic map of (A) pComb3/green and (B) pComb3/white. Fragments not to scale.

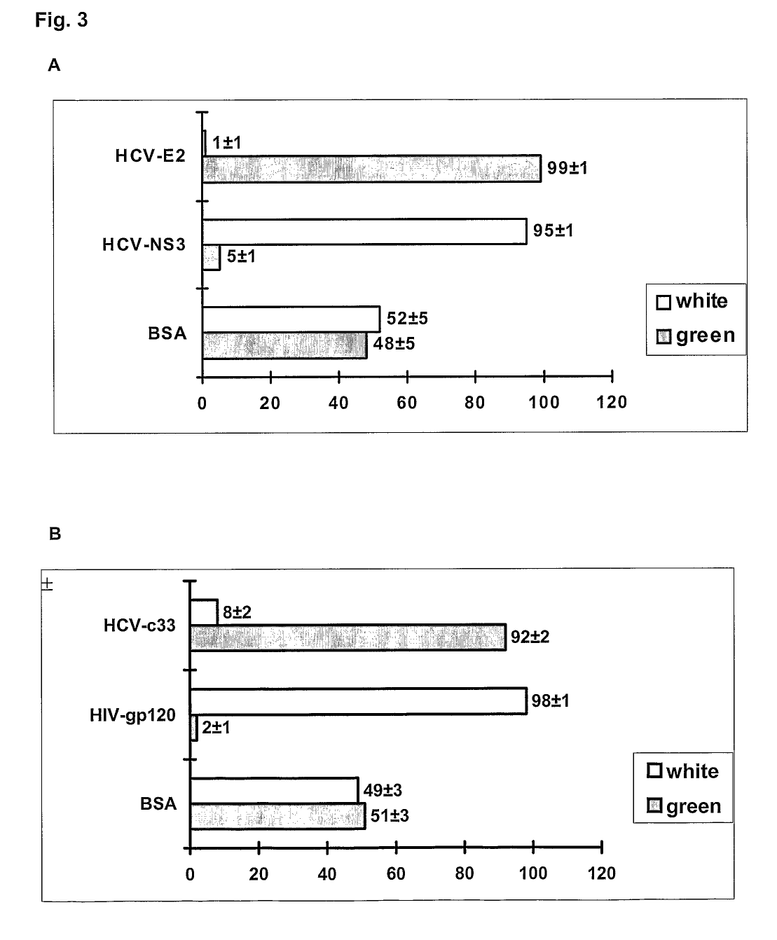

FIG. 3: Selection by immunoaffinity against antigens (HCV-E2 and HCV/NS3) fixed on solid phases of mini library A; and against antigens (HCV-c33 and HIV/gp120) fixed on solid phases of mini library B.

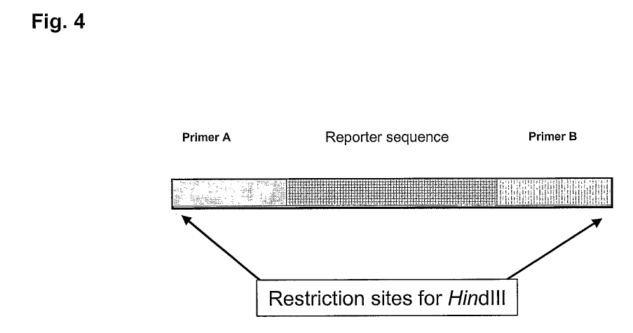

FIG. 4: diagram of the reporter sequence inserted in the HindIII site of the pComb/green vector.

To demonstrate that the insertion in a strategic point of extraneous segments of DNA in a phage vector for phage display does not disturb the production and the binding efficiency of the CDCLS-phage-DNA (CAFD) complex, the authors constructed a pair of phage vectors that contain in their structure the coding gene for a bacterial acid phosphatase (Burioni, Plaisant et al. 1997) (Burioni, Plaisant et al. 1995) that in one vector (green) is active whilst in the other one (white) is inactivated. This approach has allowed to insert this gene in different positions, enabling the authors immediately to distinguish the bacterial colonies that contained a phagemid with the insert. Moreover, this enables the experimenter to distinguish the two species simply by observing the plated colonies in a suitable modified medium. The vector used (but obviously, any other vector can be used) was the vector pComb3 (Barbas, Kang et al. 1991), extensively used both for cloning antibodies, and for cloning other oligopeptide ligands (Barbas, Crowe et al. 1992) (Williamson, Burioni et al. 1993). In detail, a pair of vectors was obtained constructing from pComb3 a new phagemid (pComb3/green) containing a fragment of DNA that encodes for the acid phosphatase of Providencia stuartii (Burioni, Plaisant et al. 1995). From pComb3/green the version with the inactivated gene was obtained (pComb3/white) in which the phosphatase gene was modified with a frameshift mutation that inactivated the product of the gene. E. coli cells containing the phagemid in white version can easily be differentiated from those containing the green version using an assay on semisolid medium. Indeed, in an appropriate medium, the presence of the DNA fragment that encodes for phosphatase provides E. coli with a brilliant green phenotype, very easy to differentiate from the cells that contain the inactivated version of the gene (FIG. 1).

As described below, the authors demonstrated that the insertion of a DNA fragment in this specific site does not disturb either the production of the phage, or its assembly, or the ability of the antibody (that serves as CDCLS)-phage-DNA (CAFD) complex to bind efficiently to an antigen. The phagemids are produced in identical fashion, and in a manner that is not different from the parental vector pComb3, once the bacterial cells are infected. Lastly, the phenotype is strictly correlated to the genotype, thereby confirming the stability of the antibody CDCLS-phage-DNA complex (CAFD) and its adequacy for the purposes illustrated herein.

To further confirm the efficiency and stability of the system, the authors demonstrated the capability of the CAFD complex to bind the specific ligand constructing two “mini-libraries” (A and B) containing antibodies of given specificity and demonstrating that selected colonies effectively corresponded to bacteria harbouring the correct CDCLS. Lastly, the authors replaced the phosphatase coding sequence by amplifiable DNA sequences and demonstrated that a specific amplification is obtained after the CDCLS binds to the analyte fixed on solid phase.

### Construction of the Vectors

The construction of the vectors is described in detail in the materials and methods part in the experimental protocol. Briefly, the resulting pComb3/green vector is a derivative of pComb3 with a size of 6.4 Kb which maintains all restriction sites of parental vector and which gives to E. coli cells transformed with this vector a brilliant green phenotype in TPA/MG culture medium (Satta, Grazi et al. 1979), (Fig. A). pComb3/white is derived from pComb3/green but the reading frame of the coding gene for the P. stuartii phosphatase was destroyed by digestion with HindIII, subsequent filling of the protruding ends and religation; pComb3/white has the same characteristics and dimensions as pComb3/green, but does not give the green colour to E. coli colonies transformed with it when they are grown on plates containing the TPA/MG culture medium. pComb3/green and pComb3/white vectors are schematically illustrated in FIG. 2. Subsequently, some regions of the fragment containing the alkaline phosphatase gene were replaced with target of synthetic DNA synthesised in vitro. The insertion of such sequences, described below, was carried out using current molecular biology techniques.

### Production of the CDCLS (or Antibody)-Phase-DNA Complex

The first experimental issue to resolve was whether a DNA insert with a size of about 1.0 Kb, positioned in the selected point, could disturb phage production or lead to an incorrect encapsulation of the DNA. For this reason, E. coli cells were infected according to already described procedures (Barbas, Crowe et al. 1992) with 1×10⁷ phages with a ratio of about 1:1 between pComb3/green:pComb3/white and from the infected cells, phage production was carried out as described previously (Burioni, Plaisant et al. 1997). An infected portion of E. coli cells was plated in TPA/ampicillin plates (100 μg/ml) where only the cells containing pComb3/white or pComb3/green were able to grow. The total number of phage used for the infection and the number of colonies were counted, demonstrating as indicated by the green-white ratio, the correct proportion of the two species in the phage population. The following morning (18 hour from the infection) the phages were prepared as described in the materials and methods section by precipitation with PEG and were used to infect new bacterial cells (Burioni, Plaisant et al. 1997). If the production of the two forms had been identical, the proportion in the phages produced the following day should have been the same as the one of the previous afternoon. A minimal unbalance, during the production time, would have led to an evident prevalence of one of the two forms. To evaluate this aspect, the bacterial cells infected with the phage just produced (as described in the materials and methods section) were plated on MG/TPA agar and the white and green cells were counted. The results of the experiments conducted four times in totally independent fashion are shown in Table 1. The proportion of the two families of phages remained substantially equal after an amplification cycle demonstrating the absence of a replication advantage of one of the two forms which might have been introduced by the insertion of this gene in the phagemid. The absolute value of phages generated during these experiments was around 1×10¹³/ml, which is similar to what is usually obtained with the pComb3 vector not modified in similar amplification procedures (Williamson, Burioni et al. 1993). These data confirm that the insertion of a DNA fragment in the indicated position does not disturb phage production and assembly.

TABLE 1
Amplification of mixed pComb3/green
and pComb3/white populations.

| experiment # | % input ratio (g/w) | % output ratio (g/w) |
|---|---|---|
| 1 | 45/55 | 52/48 |
| 2 | 52/48 | 50/50 |
| 3 | 44/56 | 47/53 |
| 4 | 56/44 | 54/45 |

The ratio is expressed as green/white

The next step was the demonstration that the phages remain stable, and that a given DNA fragment (in this case, containing the native or modified phosphatase, which provides the bacterium that contains it with an easily identifiable phenotype) remains stably associated to the genes of the CDCLS antibody, so consequently it is able to constitute a stable CDCLS antibody-phage-DNA (CAFD) complex. To achieve this objective, for each experiment ten white colonies and ten green colonies were isolated, grown, and the phagemidic DNA was prepared by miniprep (Maniatis 1988). The association between the heavy chain of the antibody and the (active or non active) phosphatase was confirmed by DNA sequencing, as expected. This confirmed the stability of the binding between the DNA reporter and the compound having binding capability (in this case a human antibody) and hence the adequacy of the approach.

### Mini-Library Assay

To determine whether the presence of an exogenous DNA fragment, stably bound to a specific binding compound, would interfere with the binding capability of the compound itself, two artificial mini-libraries were constructed. This was obtained by cloning in the two vectors, one containing the active phosphatase and the other the inactive phosphatase, alternatively the coding genes for a human Fab directed against the glycoprotein E2 of HCV/E2 (Burioni, Plaisant et al. 1998) or directed against the NS3 antigen of the same virus (Plaisant, Burioni et al. 1997). A mini-library was prepared with a 1:1 mixture of pComb3/white-Fab(HCV/NS3) and pComb3/green-Fab(HCV/E2). The selection of this mini-library against an antigen fixed on solid phase produced a population of colonies with the green phenotype if the antigen on solid phase was E2, with the white phenotype if the antigen on solid phase was NS3. The second mini-library was constructed in opposite fashion, with a 1:1 mixture of pComb3/white-Fab(HCV/E2) and pComb3/green-Fab(HCV/NS3). In this case, the expected results are opposite to those illustrated previously. The two artificial mini-libraries were then subjected to an immunoselection cycle by panning against the two relevant antigens (HCV/NS3 and HCV/E2) and against a negative control, bovine serum albumin (BSA). The results shown in FIG. 3 clearly indicate that in all cases, phages selected by means of immunoaffinity have the expected phenotype, thereby demonstrating the selection of the specific DNA sequence, which may thus be exploited to demonstrate, indirectly, the binding of the CDCLS antibody. Further studies conducted by preparation of the phagemidic DNA, digestion with restriction enzymes and sequencing, confirmed that the genome structure of the CDCLS-phage-DNA complex was exactly as expected. The correct selection of the CDCLS-phage-DNA complexes was also confirmed by transforming the vectors into phagemids able to produce corresponding antibody fragments (Fab) in soluble form: all transformed clones have produced Fab with the expected specificity. The reliability of the production system of the CDCLS-phage-DNA complex was also demonstrated observing the selection against an antigen not recognised by the two antibodies mounted in the complexes used. As expected, the selection against an irrelevant antigen like BSA produced a population of phages having to an equal extent the two phenotypes, confirming the unbiased production of the two vector forms. Naturally, the absolute number of phages was very different when the selection took place against a relevant antigen like HCV/NS3 or HCV/E2 (the phages eluted from a well in this case were between 10⁶ and 10⁷), or in the case of the irrelevant antigen (around 10⁴). These values are substantially identical to those obtained during common experiments of phage selection by immunoaffinity.

### Demonstration of the Binding of the CDCLS-Phage-DNA Complex by Amplification of a DNA Fragment Inserted in the Genome of the Phagemid

Using a molecular analysis program (Oligo 4.0), two DNA fragments were designed, containing a random specific sequence of bases, with a content in G+C equivalent to A+T content, and stable (Rychlik and Rhoads 1989; Rychlik, Spencer et al. 1990). The fragments, constituted by two synthetic DNAs hybridised in liquid phase, were constituted by three separate sequences:

i) a “primer A” region at the 5′ end identical for both fragments,
ii) a central region (“reporter”) different for each of the fragments and
iii) a “primer B” region at the 3′ end identical for both fragments (FIG. 4).

At the 5′ and 3′ ends were inserted two restriction sites recognised by the Hind III enzyme, distanced by a spacer from the terminal of the DNA to optimise digestion by the restriction enzyme. The synthetic DNA fragments were cut with Hind III and were inserted by ligation with T4 DNA ligase (Maniatis 1988) in the pComb3/green vector, cut with the same enzyme and dephosphorylated. The insertion of the DNA fragment was identified against the background by plating the result of the transformation of the ligation in TPA/MG-ampicillin. Two different constructs were produced, each containing the genes of one of the two antibodies (anti E2 and anti NS3) and a DNA fragment with the two primers identical but with different reporter sequences. The construct was sequenced, characterised by digestion with restriction enzymes, and the phage DNA detection was revealed by amplification of the synthetic DNA fragment inserted as described above. Amplification was conducted using 40 cycles (94° C. for 15 seconds, 54° C. for 15 seconds and 72° C. for 20 seconds) and the primers corresponding to the ends of the synthetic DNA fragment were used. The presence of an amplimer was demonstrated by polyacrylamide gel. Using the constructs described above, E. coli cells were transformed and used to prepare a phage suspension according to methods already mentioned above. Through an amplification reaction already described above, obviously considering the polarity of the genome with single filament of the phage DNA, it was possible to demonstrate the presence of the synthetic DNA inside the phage suspension using 1 μl of suspension and introducing at the start of the PCR reaction a 30 second denaturation step at 94° C. After verifying the presence of the synthetic DNA in the phage, two mini-libraries were constructed, which were used in identical fashion to the one described above. The presence of the two species of phages was demonstrated by subjecting 1 μl of eluate to the amplification described above, and demonstrating the presence of one of the two synthetic DNAs using one of the primers biotinylated and by means of specific hybridisation in liquid phase with plates able to bind DNA covered with specific probes for the label sequence of only one of the two DNAs. The binding of the amplified DNA with specific probes was demonstrated by immunoenzymatic assay and measure of the optical density with a spectrophotometer. The results confirmed the detection of the phosphatase activity as already observed in the assay conducted with the mini-libraries.

The results demonstrated that the construction of a CDCLS-phage-DNA complex generates a reagent in a reproducible, fast and economical fashion. The complex obtained with the method of the invention can be used efficiently to demonstrate the binding to a specific ligand by the detection of the DNA. The complex is used to reveal the presence of an analyte, having a specific ligand available. The described CDCLS-phage-DNA complex is used not only in a single form, but also using simultaneously different constructs and the product of the amplification can be quantified using solid supports (chips) whereto have been fixed specific DNA sequences, complementary to the different label sequences inserted in the synthetic DNA inserted in the reporter sequences of the phagemid that constitutes the genome of the artificial bacterial virus.

The method allows the rapid, economical and simultaneous detection of the presence of a potentially unlimited number of analytes, either directly fixed on an activated binding surface, or fixed by means of a sandwich with another CDCLS fixed on an appropriate solid phase.

In addition to the detection of the presence of specific ligands, the method can be exploited to detect phage sub-populations in artificial mini-libraries, useful to evaluate the in vivo effectiveness of pharmaceutical preparations that are potentially usable as vaccines (Parren, Fisicaro et al. 1996). This is particularly relevant for pathogenic agents lacking adequate animal models (such as the acquired immune deficiency virus, HIV, or the hepatitis C virus, HCV) and many important agents causing severe illnesses.

### Materials and Methods

### Bacterial Strains, Vectors and DNA Fragments.

E. coli XL1-Blue bacterial strain (Stratagene, La Jolla, Calif.) was acquired from Stratagene. pComb3 and the gene of the P. stuartii acid phosphatase have been described in the literature (Barbas, Kang et al. 1991) (Burioni, Plaisant et al. 1995).

Construction of the pComb3/Green and pComb3/White Vectors

The two vectors were constructed using standard molecular biology techniques (Sambrook, Fritsch et al. 1989). All reagents used in this study were obtained from Boheringer Mannheim, Germany. In detail, the insert containing P. stuartii acid phosphatase gene was obtained digesting pPho2 vector (Burioni, Plaisant et al. 1995) with SpeI and SmaI restriction endonuclease (ER). The correctly sized DNA fragment was purified from gel and the 3′-terminal ends were made blunt with Klenow DNA polymerase. This fragment (20 ng) was ligated for 2 hours at 16° C. in a total volume of 20 μl, at the Sac1 site of the pComb3/B vector (Burioni, Plaisant et al. 1997) (after blunting the 5′ terminal ends with T4 DNA polymerase). The ligation products were used to transform by electroporation electrocompetent E. coli cells that were plated on triptose phosphate agar/methyl green (TPA/MG) (Satta, Grazi et al. 1979) containing ampicillin (100 μg/ml). Subsequently, the green colonies that presumably contain the phosphatase gene were drawn and through an analysis conducted with restriction endonuclease, it was possible to determine the presence and the orientation of the fragment derived from pPho2. One of the clones containing the phosphatase gene with the correct orientation that gave to E. coli a green phenotype on TPA/MG medium was called pComb3/green and subsequently used. The pComb3/white was obtained from pComb3/green by destroying the correct reading frame with a mutation able to destroy the phosphatase activity (R.B., unpublished data): pComb3/green was digested with HindIII ER (able to cut only inside the phosphatase gene) and the DNA thus linearised was blunted and ligated again and used to transform electrocompetent E. coli cells which were then plated on TPA/MG-ampicillin plates. Ten white colonies were drawn from the plate and it was demonstrated that the mutated phosphatase gene was present in all of them. From these colonies, a clone was selected, which was called pComb3/white and used for the subsequent experiments.

Production of the Phage from DNA Phagemid.

The phages were produced starting from bacteria transformed with the phagemid as described by Barbas et al. (Barbas, Kang et al. 1991). Briefly, 100 μl of electrocompetent E. coli XL1-Blue cells were electrotransformed (Barbas, Kang et al. 1991) with about 10 pg phagemid. After transformation, 2 ml of SOC medium were added (Barbas, Bain et al. 1992) and the culture was left in agitation at 220 rpm for 1 hour at 37° C.; subsequently, 10 ml of SB medium were added (30 g tryptone, 20 g yeast extract, 10 g MOPS per litre, pH 7) containing ampicillin (20 μg/ml) and tetracycline (10 μg/ml). The culture is grown for 1 hour at 37° C. in agitation at 250 rpm. This culture was added to 100 ml of SB containing ampicillin (50 μg/ml), tetracycline (10 μg/m), then the helper phage VCS-M13 (10¹² pfu) was added and the culture was left in agitation for 2 more hours. After adding kanamycin at the final concentration of 70 μg/ml the culture was incubated overnight at 37° C. The supernatant was clarified by centrifuging at 4° C. The phage was precipitated adding polyethylene glycol 8000 4% and NaCl 3% (final concentrations), incubated on ice for 30 minutes, and centrifuged. The phage pellet was resuspended in 2 ml PBS (phosphate 50 mM, pH 7.2, NaCl 150 mM)/bovine serum albumin 1% (BSA) and centrifuged for 3 minutes to eliminate detritus, and lastly transferred into new tubes and if necessary preserved at −20 C°.

The same procedure was carried out for the production of phage from stock, but instead of the transformation an appropriate quantity of phage was used to infect 200 μl of E. coli cells OD₆₀₀=1. The phage and the cells were incubated for 15 minutes at ambient temperature, and subsequently were added 10 ml SB containing ampicillin (20 μl/ml) and tetracycline (10 μl/ml). Thereafter, the procedure followed is identical.

Titre of the Colony Forming Units (cfu).

The phagemids that were packed in the virions are able to infect E. coli and to form colonies on selective plates. The phages (the packed phagemids) were diluted in SB (dilutions of 10³, 10⁶, and 10⁸), and 1 μl was used to infect 50 μl of E. coli XLI-Blue OD₆₀₀=1, grown in SB containing tetracycline (10 μg/ml). The phage and the cells were incubated at ambient temperature for 15 minutes, then 10 μl were plated directly on LB/ampicillin plates (to determine the absolute number of phages) and in parallel on TPA-MG/ampicillin plates (to determine the white/green ratio).

### Panning of the Combinatorial Library to Select the Phases Binding the Antigen.

The panning procedure was performed as described by Burton et al. (Burton, Barbas et al. 1991). Four wells of a microtitre plate (Costar) were coated overnight at 4° C. with 100 ng of antigene in PBS (25 μl). The wells were washed 5 times with water and blocked by covering each well completely with BSA 3% in PBS and incubating the plate at 37° C. for 1 hour. The blocking solution was removed and to each well were added 50 μl of a fresh phage preparation (typically 10¹¹ cfu), the plate was incubated for 2 hours at 37° C. The phage was removed and the plate was washed once with water. Each well was then washed 10 times with PBS/Tween20 0.5% for 1 hour at ambient temperature. The plate was washed an additional time with distilled water and the bound phage was eluted adding 50 μl of elution buffer (HCL 0.1 M, brought to pH 2.2 with solid glycine) to each plate; the plate was left at ambient temperature for 10 minutes. The elution buffer was pipetted up and down a few times, removed and neutralised with 3 μl of Tris base 2M for 50 μl of elution buffer. The eluted phage was used to infect 2 ml of a fresh culture of E. coli XL1-Blue (OD₆₀₀=1) for 15 minutes at ambient temperature, 10 ml of SB containing carbenicillin (20 μg/ml) and tetracycline (10 μg/ml). Portions equal to 20, 1 and 0.1 μl were drawn to be plated on LB/ampicillin plates and to determine the number of phages (the packed phagemids) eluted from the plate. Similar portions were plated in parallel on TPA/MG plates to determine the phenotype of the colonies.

### BIBLIOGRAPHICAL REFERENCES

Baldo, B. A., Tovey, et al. (1986). J Biochem Biophys Methods 12(5-6): 271-9.
Barbas, C. F., D. Bain, et al. (1992). Proc. Natl. Acad. Sci. USA 89: 4457-4461.
Barbas, C. F., Crowe, et al. (1992). Proc. Natl. Acad. Sci. USA 89: 10164-10168.
Barbas, C. F., Kang, et al. (1991). Proc. Natl. Acad. Sci. USA 88: 7978-7982.
Bodmer, D. M. and L. X. Tiefenauer (1990). J Immunoassay 11(2): 139-45.
Burioni, R., P. Plaisant, et al. (1997). Res. Virol. 148: 161-164.
Burioni, R., P. Plaisant, et al. (1998). Hepatology 28(3): 810-4.
Burioni, R., P. Plaisant, et al. (1995). Microbiologica 18: 201-206.
Burton, D. R., Barbas, et al. (1991). Proc. Natl. Acad. Sci. USA 88: 10134-10137.
Graves, H. C. (1988). J Immunol Methods 111(2): 167-78.
Hauri, H. P. and K. Bucher (1986). Anal Biochem 159(2): 386-9.
Hendrickson, E. R., T. M. Truby, et al. (1995). Nucleic Acids Res 23(3): 522-9.
Li, H., X. Cui, et al. (1990). Proc Natl Acad Sci USA 87(12): 4580-4.
Maniatis (1988). Molecular Cloning: a laboratory manual.
Maxam, A. M. and W. Gilbert (1977). Proc Natl Acad Sci USA 74(2): 560-4.
Parren, P. W. H. I., P. Fisicaro, et al. (1996). J. Virol. 70: 9046-9050.
Plaisant, P., R. Burioni, et al. (1997). Res. Virol. 148: 165-169.
Pruslin, F. H., S. E. To, et al. (1991). J Immunol Methods 137(1): 27-35.
Rodda, D. J. and H. Yamazaki (1994). Immunol Invest 23(6-7): 421-8.
Ruan, K., S. Hashida, et al. (1986). Ann Clin Biochem 23 (Pt 1): 54-8.
Rychlik, W. and R. E. Rhoads (1989). Nucleic Acids Res 17(21): 8543-51.
Rychlik, W., W. J. Spencer, et al. (1990). Nucleic Acids Res 18(21): 6409-12.
Sambrook, J., E. F. Fritsch, et al. (1989). Molecular cloning: a laboratory manual. Cold Spring Harbour, Cold Spring Harbour Laboratory Press.
Sanger, F. and A. R. Coulson (1975). J Mol Biol 94(3): 441-8.
Sano, T. and C. R. Cantor (1991). Biotechnology (N Y) 9(12): 1378-81.
Sano, T., C. L. Smith, et al. (1992). Science 258(5079): 120-2.
Satta, G., G. Grazi, et al. (1979). J. Clin. Pathol. 32: 391-395.
Tovey, E. R., S. A. Ford, et al. (1989). Electrophoresis 10(4): 243-9.
Vogt, R. F., Jr., D. L. Phillips, et al. (1987). J Immunol Methods 101(1): 43-50.
Wedege, E. and G. Svenneby (1986). J Immunol Methods 88(2): 233-7.
Williamson, R. A., et al. (1993). Proc. Natl. Acad. Sci. USA 90: 4141-4145.
Zhou, H., R. J. Fisher, et al. (1993). Nucleic Acids Res 21(25): 6038-9.

1. A complex able to detect an analyte (CRA) comprising a particle expressing on its outer surface a compound having specific binding capability (CDCLS) for the analyte and stably including at least one nucleic acid reporter sequence being univocally associated to the CDCLS.

2. The complex according to claim 1 wherein the particle is a recombinant particle.

3. The complex according to claim 2 wherein the recombinant particle is a recombinant virus particle.

4. The complex according to claim 3 wherein the recombinant virus particle is a recombinant bacterial phage particle.

5. The complex according to any of previous claims wherein the nucleic acid reporter sequence encodes for a detectable marker.

6. The complex according to claim 4 wherein the detectable marker is a phosphatase or a beta-galactosidase.

7. The complex according to claims 1-3 wherein the nucleic acid reporter sequence is flanked at its 5′ end by a first primer sequence, and at its 3′ end, by a second primer sequence.

8. The complex according to any of previous claims wherein the CDCLS is an antibody, or a functional fragment thereof obtained by synthetic or recombinant procedures, or a bispecific antibody.

9. The complex according to claims 1-7 wherein the CDCLS is a non antibody protein, a peptide, even in multimeric form and/or made by modified or non natural amino acids.

10. A recombinant or combinatorial library comprising a collection of the complexes according to any of previous claims wherein each CDCLS is associated to a different nucleic acid reporter sequence.

11. The recombinant or combinatorial library according to claim 10 wherein the first primer sequence and the second primer sequence are each hybridisable to a first primer and to a second primer under high stringency conditions, respectively.

12. A process for constructing a complex according to claim 1-9 comprising the steps of:
a) inserting into an host cell an appropriate recombinant vector comprising coding sequences for the CDCLS linked to appropriate sequences to direct its expression on the outer surface of a recombinant virus particle;
b) transforming cells as obtained in a) with a packageable genome containing the nucleic acid reporter sequence, and
c) infecting said transformed cells with a helper virus able to rescue a recombinant virus particle expressing on its outer surface the CDCLS and stably including at least one nucleic acid reporter sequence.

13. A process for constructing a complex according to claim 1-9 comprising the steps of:
a) transforming an host cell an appropriate recombinant viral vector comprising: i) coding sequences for the CDCLS linked to appropriate sequences to direct its expression on the outer surface of a recombinant virus, ii) nucleic acid sequences allowing the encapsulation of the vector inside the recombinant virus particle and iii) the nucleic acid reporter sequence;
b) infecting said transformed cells with a helper virus able to rescue a recombinant virus particle expressing on its outer surface the CDCLS and stably including at least one nucleic acid reporter sequence.

14. The process according to claim 13 wherein the appropriate recombinant viral vector consists in a collection of different vectors, each one comprising a given CDCLS coding sequence univocally associated to a given nucleic acid reporter sequence.

15. Method for detecting an analyte in a sample comprising the steps of:
a) incubating the sample with a solid phase specific for the analyte in such conditions that, if present, the analyte binds to the solid phase;
b) incubating the solid phase whereto is bound the analyte, if present, with the CRA as claimed claims 1-9 in conditions that, if present, the analyte binds to the CDCLS of the CRA;
c) separating the solid phase-analyte-CRA complexes from non bound CRAs;
d) detecting the reporter sequences present in the solid phase-analyte-CRA complex.

16. Method as claimed in claim 15 wherein the detection of the reporter sequences is made by an amplification thereof.

17. Kit for detecting an analyte in a sample comprising the complex as claimed in one of the claims 1-9.
